(19) United States
(12) Patent Application Publication
(10) Pub. No.: US 2015/0196752 A1
(43) Pub. Date: Jul. 16, 2015

(54) EPICARDIAL LEAD PLACEMENT APPARATUS, SYSTEMS, AND METHODS

(71) Applicant: Intermountain Invention Managment, LLC, Salt Lake City, UT (US)

(72) Inventors: Thomas Jared Bunch, South Jordan, UT (US); Troy Jesse Orr, Draper, UT (US)

(21) Appl. No.: 14/420,435

(22) PCT Filed: Aug. 9, 2013
(86) PCT No.: PCT/US2013/054327

§ 371 Date: Feb. 9, 2015

Related U.S. Application Data
(60) Provisional application No. 61/682,131, filed on Aug. 10, 2012.

(51) Int. Cl. A61N 1/05

(52) U.S. Cl. CPC A61N 1/059

(57) ABSTRACT

An anchoring assembly can be used to electrically couple an electrical lead with the heart of a patient. The anchoring assembly can include a guidewire and a magnetic anchor. At least a distal portion of the anchoring assembly can be sized to be inserted between the pericardium and the epicardium, and the magnetic anchor can magnetically interact with a magnetic guide that is positioned at an interior of the endocardium to hold the anchoring assembly against the epicardium.

### CROSS-REFERENCE TO RELATED APPLICATIONS

This application claims the benefit of U.S. Provisional Patent Application No. 61/682,131, titled EPICARDIAL LEAD PLACEMENT APPARATUS, SYSTEMS, AND METHODS, which was filed on Aug. 10, 2012, the entire contents of which are hereby incorporated by reference herein.

### BACKGROUND

Apparatus, systems, and methods for epicardial lead placement suffer from one or more drawbacks. These can be resolved, remedied, ameliorated, or avoided by certain embodiments described herein.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A is a schematic cross-sectional view of a portion of a patient that includes the heart of the patient and a partial perspective view of a portion of a system for positioning an electrical lead relative to the heart, wherein the portions of the system that are shown include a guiding assembly, which includes a steering catheter having a magnetic guide at a tip thereof, and a syringe for the delivery of contrast and are depicted in an early stage of an illustrative lead placement procedure;

FIG. 1B is a view such as that of FIG. 1A showing another stage of the procedure, wherein a needle portion of the syringe remains positioned at a space between the epicardium and the pericardium of the heart, and wherein a guidewire has been advanced through the needle portion into the pericardium;

FIG. 1C is a view such as that of FIG. 1A showing another stage of the procedure, wherein the needle has been removed and replaced with an introducer sheath and dilator;

FIG. 1D is a view such as that of FIG. 1A showing another stage of the procedure, wherein the guidewire has been removed and replaced with an anchoring assembly that includes one or more guidewires that are different from that shown in FIG. 1C and a magnetic anchor;

FIG. 1E is a view such as that of FIG. 1A showing another stage of the procedure, wherein the tip of the magnetic guide portion of the steering catheter has been moved to reposition the magnetic anchor;

FIG. 2A is an enlarged view taken along the view line 2A of FIG. 1E that depicts the magnetic guide and the magnetic anchor in greater detail at another stage of the procedure in which an elongated body that includes an electrical lead has been advanced toward the magnetic anchor;

FIG. 2B is an enlarged view such as that of FIG. 2A showing another stage of the procedure, wherein a portion of the anchoring assembly has been reconfigured to orient a fastener for insertion into the epicardium;

FIG. 3A is a partial perspective view of the stage of the procedure shown in FIG. 2A;

FIG. 3B is a partial perspective view of the stage of the procedure shown in FIG. 2B;

FIG. 3C is a partial perspective view of another stage of the procedure in which the fastener is being advanced into the myocardium;

FIG. 3D is a partial perspective view of another stage of the procedure in which the fastener has been advanced into the myocardium and in which the guidewire and the magnetic anchor have been removed;

FIG. 4 is a schematic perspective view of an embodiment of a guiding assembly suitable for use with the system of FIG. 1, wherein the guiding assembly includes a steering catheter;

FIGS. 5A-5C are cross-sectional views of an embodiment of a steering catheter that is compatible with the system of FIG. 4, wherein the steering catheter is shown in various orientations to which it can be moved via a steering wire;

FIG. 6A is an enlarged view of a portion of another system for positioning an electrical lead relative to the heart at an intermediate stage of another example of a lead placement procedure, wherein the view of FIG. 6A is similar to that shown in FIG. 2A, and wherein the system includes an embodiment of a pushing element for advancing a magnetic anchor through an introducer sheath and to a desired position at which the magnetic anchor can interact with a magnetic guide;

FIG. 6B is another view of the system of FIG. 6A at a later stage of the procedure, wherein the pushing element has been retracted over a guidewire and an embodiment of a lead assembly is being advanced over the same guidewire toward the magnetic anchor;

FIG. 6C is another view of the system of FIG. 6A at a further stage of the procedure, wherein a fastener portion of the lead assembly is being advanced into the epicardium at a position adjacent to the magnetic anchor;

FIG. 7A is a partial perspective view of another embodiment of a system for positioning an electrical lead relative to the heart, wherein the view is similar to that depicted in FIG. 3C, and wherein a stage of an electrical lead placement procedure is shown with another embodiment of a fastener being advanced into the epicardium;

FIG. 7B is a partial perspective view of another stage of the procedure depicted in FIG. 7A, wherein the fastener has been advanced into the epicardium and a guidewire and magnetic anchor have been removed;

FIG. 8A is a partial perspective view of another embodiment of a system for positioning an electrical lead relative to the heart, wherein the view is similar to that depicted in FIG. 3C, and wherein a stage of an electrical lead placement procedure is shown with another embodiment of a fastener being advanced through the epicardium into the myocardium;

FIG. 8B is a partial perspective view of another stage of the procedure, wherein the fastener is embedded in the myocardium and wherein a guidewire has been split apart from a magnetic anchor; and

FIG. 8C is a partial perspective view of another stage of the procedure, wherein the guidewire and the magnetic anchor have been withdrawn from the patient.

### DETAILED DESCRIPTION

The present disclosure relates generally to epicardial lead placement, such as devices, systems, and methods for the placement of epicardial leads used in any suitable cardiac procedure, such as, for example, cardiac resynchronization therapy. Accordingly, some embodiments are used for the placement of pacemaker epicardial leads. In various implementations, epicardial leads may be placed via minimally invasive procedures. In various embodiments, the procedures may be used to temporarily electrically couple an electrical lead wire with a position on the epicardium for positioning and/or testing. In other or further embodiments, the procedures may be used to affix, or fixate, the electrical lead wire to the epicardium for longer term use. Further discussion of various embodiments is provided below with reference to the drawings.

Certain embodiments can be particularly useful for cardiac resynchronization therapy. Such therapy is a well-validated approach for improving heart failure symptoms and heart failure class in patients who have moderate to severe heart failure. The procedure generally involves implantation of transvenous pacemaker leads in the right and left ventricles. Favorable long-term outcomes are typically dependent on a good lead position in the left ventricle, which can allow true synchronization of the ventricles. Left ventricular (“LV”) lead placement can require coronary sinus cannulation and advancement of the lead into a venous tributary, which can require considerable operator skill and experience as well as favorable anatomy. In particular, coronary sinus anatomy is highly variable, and it can also be influenced by prior cardiac surgery and/or scarring. In addition, the location of the phrenic nerve and regional epicardial scarring can impact the suitability of the LV lead placement. Basal locations for the lead placement are also possible and, in some situations, may even be preferable to typical LV lead placement. In some instances, basal locations may be less stable.

For a significant fraction of patients, an adequate LV lead position cannot be found. Such patients are generally referred to cardiac surgery for an epicardial lead placement, for example, via an additional procedure and a highly invasive thoracotomy. Accordingly, minimally invasive procedures for placing leads, such as described herein, can be particularly advantageous in this context.

Many embodiments described herein may be employed with little or no alteration of existing epicardial leads. In other embodiments, specialized epicardial leads may be provided. In either case, the epicardial leads may be placed through a minimally invasive cannulation of the pericardial sack. In some instances, the pericardial sack may be cannulated via techniques commonly performed by cardiologists and/or electrophysiologists, such as for treatment of tamponade, diagnostic assessment of a pericardial effusion, or epicardial ablation procedures.

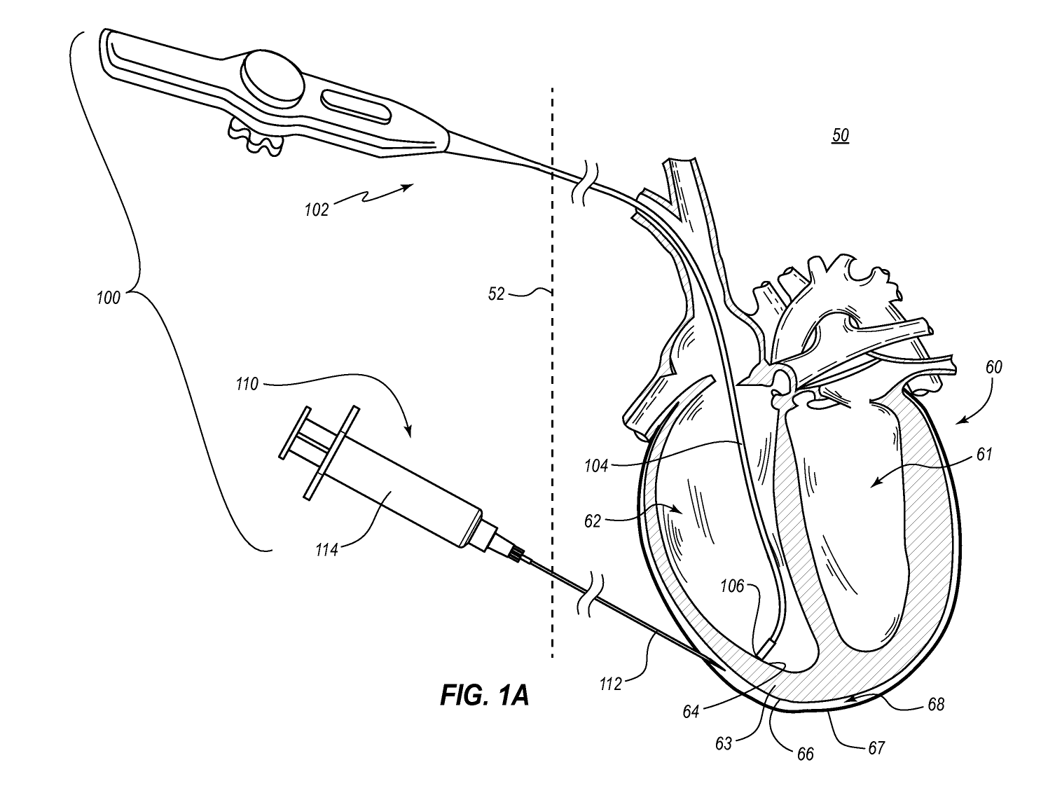

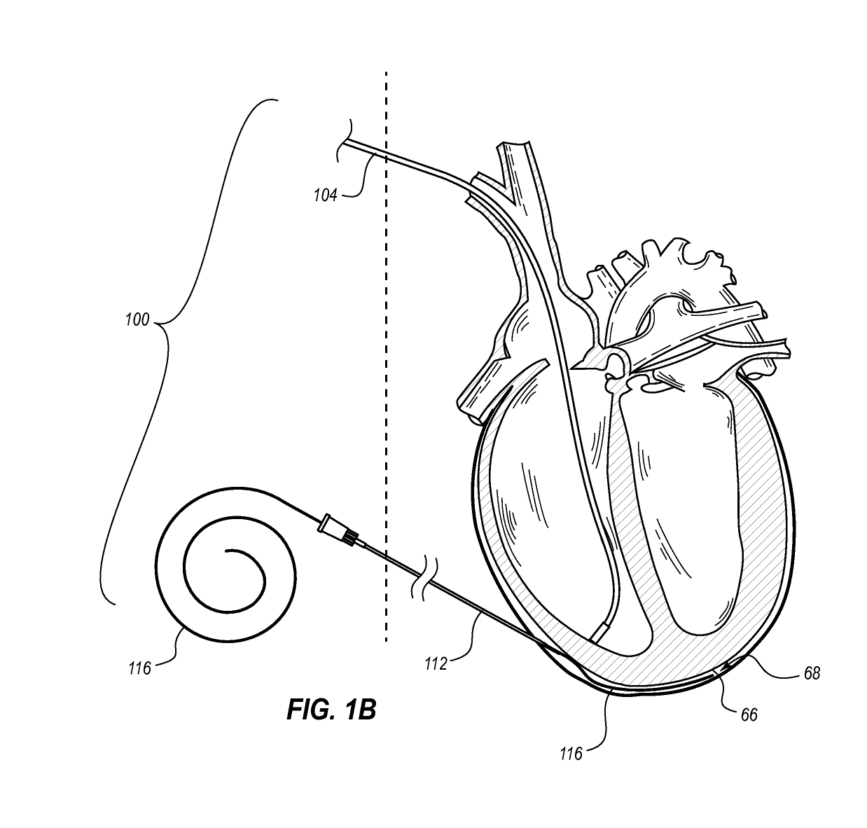

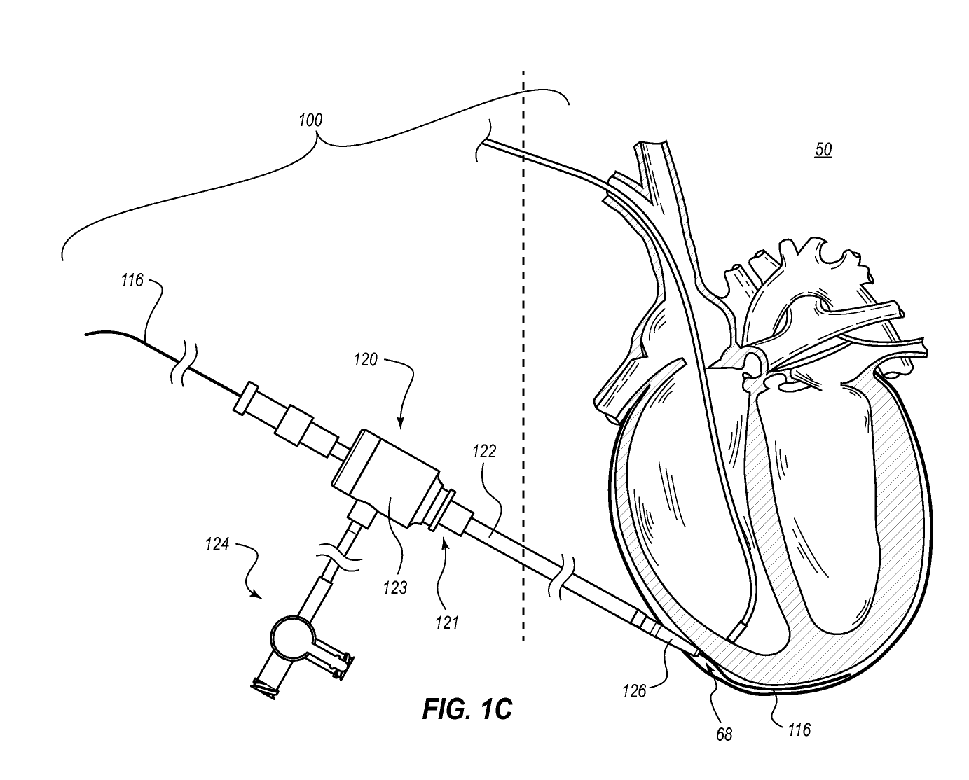

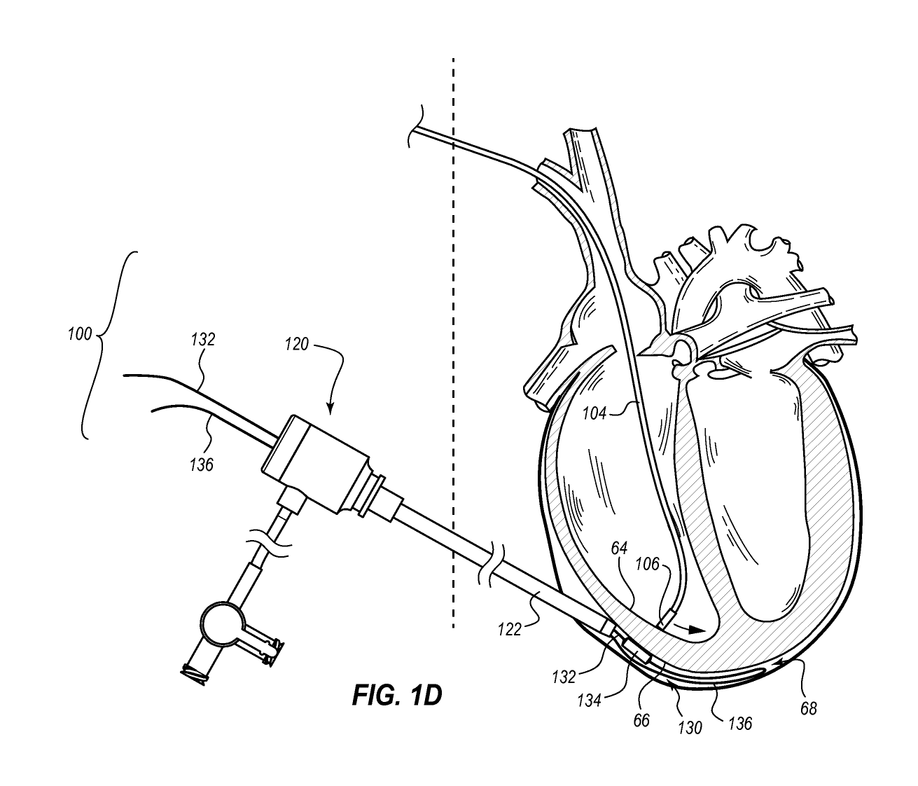

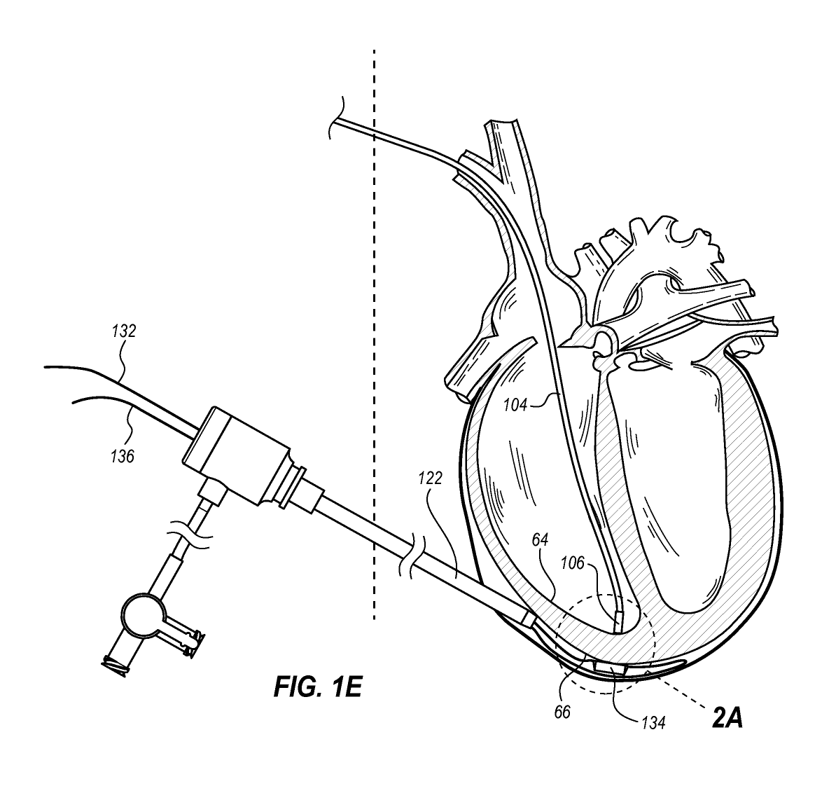

FIGS. 1A-3C illustrate various stages of a procedure for electrically coupling an electrical lead with the heart of a patient, and also illustrate various components of an embodiment of a system for electrically coupling an electrical lead with a heart. In some embodiments, the coupling can be temporary, such as for positioning the lead or testing one or more target sites at which to deliver electrical charges to the heart. In other embodiments, the coupling can be more long term (e.g., permanent), such as for prolonged use of the lead with a pacemaker or other electrical controller or signal delivering device.

The term “couple” and variants thereof are used in their ordinary sense, and include any suitable form of interaction between two or more entities, including mechanical, electrical, magnetic, electromagnetic, fluid, and thermal interaction. The terms connote some form of connection or interaction, although two components may be coupled to each other without being in direct contact with each other. Accordingly, as discussed further hereafter, different components may be magnetically coupled with each other across a wall of the heart, such as when one magnetic component aligns with and/or tracks the position of another magnetic component.

FIG. 1A illustrates an early stage of a procedure for coupling an electrical lead with a heart 60 of a patient 50. A skin surface 52 of the patient is shown schematically as a broken line. The heart 60 includes a plurality of chambers, with the left ventricle 61 and the right ventricle 62 shown. A wall 63 of the heart 60 is also shown. As is known, wall 63 can include the endocardium 64 and the epicardium 66. The heart 60 further includes the pericardium 67 and the pericardial cavity 68, which may also be described herein as a space between the pericardium 67 and the epicardium 66. The pericardium 67 is lubricated by pericardial fluid (not shown), which can cause the pericardial cavity 68 to be a difficult region within which to position an electrical lead, or which can contribute to such difficulty. For example, the pericardial fluid can cause the epicardium 66 to be slippery. Moreover, beating of the heart can further complicate placement of an electrical lead.

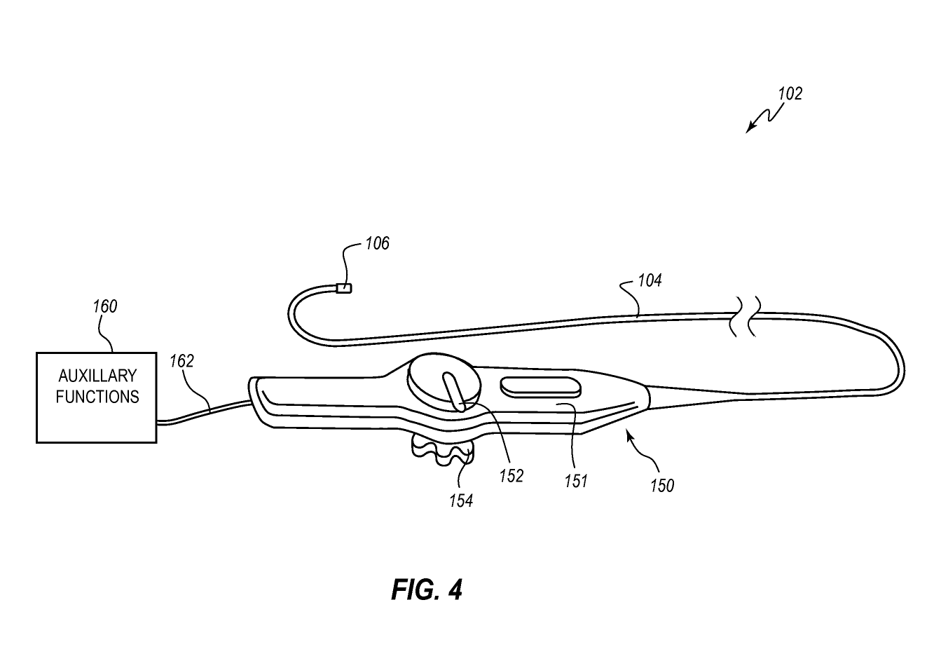

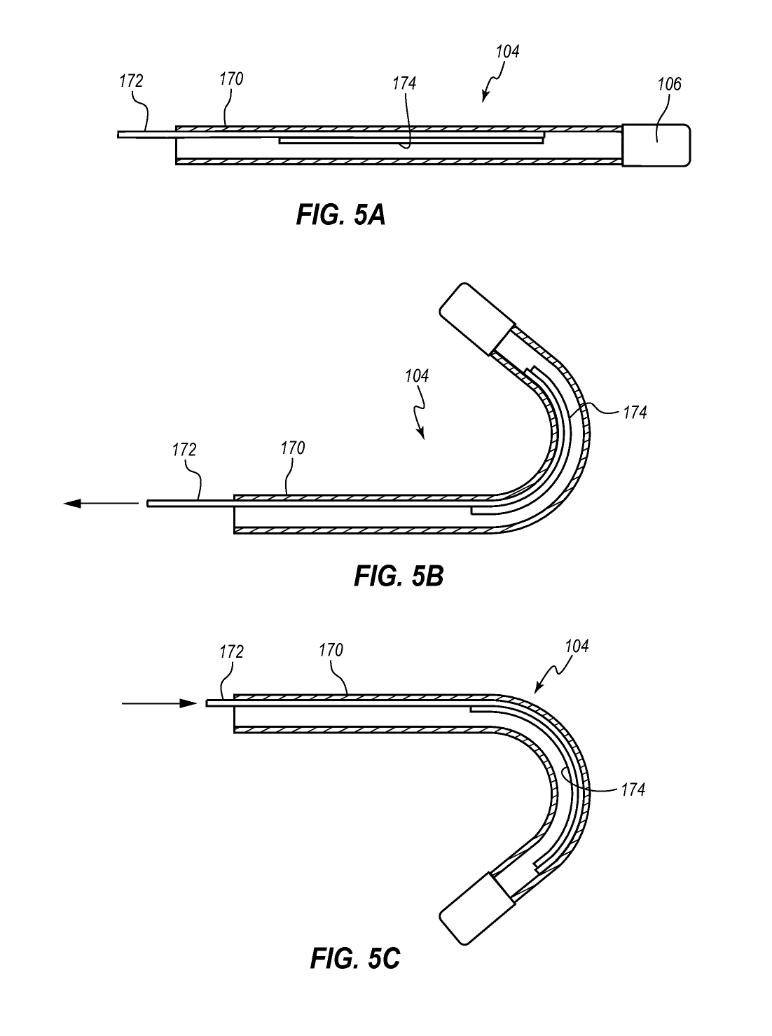

A portion of a system 100 used in the coupling procedure is shown. The system 100 includes a guiding assembly 102. In the illustrated embodiment, the guiding assembly 102 comprises a steering catheter 104, which in greater detail below with respect to FIGS. 4-5C. The steering catheter 104 can comprise a magnetic guide 106. The magnetic guide 106 can comprise any suitable magnetic material or magnetically interactive material. The terms “magnetic material” and “magnetically interactive material” are used herein in a broad sense, and can include both “magnetic sources” and “magnetically influenced materials.” The term “magnetic source” includes any suitable object or system that provides a magnetic field, such as, for example a permanent magnet, an electromagnet, or any other suitable magnetic device. The term “permanent magnet” is used herein in its ordinary sense, and includes any suitable material that provides a magnetic field in the absence of application of a magnetic field thereto, or stated otherwise, that independently or intrinsically provides a magnetic field. However, the term “permanent magnet” does not necessarily imply that the material has always provided a magnetic field or will always provide a magnetic field, since, for example, the material may have been magnetized at some prior time (e.g., via an alignment of magnetic domains), or may be demagnetized at some later time (e.g., by heating the material past its Curie temperature or via some form of degradation in which magnetic domains transition to an unaligned state). The term “magnetically influenced material” includes materials that are capable of magnetization but are not magnetized (e.g., do not independently produce a magnetic field), or that are otherwise capable of reacting to (e.g., being attracted by) or otherwise being influenced by a magnetic field. For example, magnetically influenced materials can include demagnetized, or unmagnetized, ferromagnetic and/or ferrimagnetic materials.

In various embodiments, the magnetic guide 106 comprises a magnetic material, such as, for example, iron, cobalt, nickel, ceramic composite, alnico, lanthanoid, samarium-cobalt, neodymium-iron-boron, ticonal, or rare earth materials, or any combination thereof. The magnetic material may be formed by any suitable technique such as casting, molding, sintering, stacking, etc. In further embodiments, the magnetic guide 106 can comprise a permanent magnet, such as, for example, a permanent magnet formed of any of the foregoing materials.

The steering catheter 104 can be used to position the magnetic guide 106 at or near the endocardium 64. For example, in some arrangements, the magnetic guide 106 may be positioned in abutting contact with the endocardium 64, whereas in other arrangements, the magnetic guide 106 may be in close proximity to the endocardium 64 but spaced from it. The steering catheter 104 can be configured to permit a user to readily adjust a position of the magnetic guide 106 relative to the endocardium 64, as further discussed below.

In the illustrated embodiment, the steering catheter 104 is shown extending through the vasculature of the patient 50, including the superior vena cava, and into the right ventricle 62. Accordingly, the magnetic guide 106 is positioned within the right ventricle 62. In other procedures, it may be desirable to instead position the magnetic guide 106 within the left ventricle. Any suitable method for so positioning the magnetic guide 106 is contemplated, including those known in the art.

The system can further include an insertion device 110 of any suitable variety. In the illustrated embodiment, the insertion device 110 includes a needle 112 that can be used to gain access to the pericardial cavity 68. Any suitable needle gauge is contemplated. For example, in some embodiments, the needle 112 is a 21 gauge needle.

Upon initial access of the pericardial cavity 68, the needle 112 may be coupled with a syringe 114 that includes contrast (not shown), whether before or after placement of the needle. The contrast may be injected into the pericardial cavity 68 to assist in imaging of the heart 60, various portions thereof, and/or various components of the system 100.

With reference to FIG. 1B, with the needle 112 in place, the syringe 114 can be decoupled from the needle 112. Thereafter, a guidewire 116 can be inserted through the needle 112 and into the pericardial cavity 68. Any suitable size for the guidewire is possible. For example, in some embodiments, the guidewire 116 may have a diameter of 0.035 inches. The guidewire 116 can be navigated through the pericardial space to a desired position relative to the epicardium 66.

With reference to FIG. 1C, the needle 112 can be removed from the patient 50. A percutaneous access system 120 can then be inserted into the patient 50 and into the pericardial cavity 68 over the guidewire 116. In the illustrated embodiment, the percutaneous access system 120 comprises an introducer 121. The introducer may be of any suitable variety. In the illustrated embodiment, the introducer 121 includes an introducer sheath 122 that extends from a hub 123. Also extending from the hub 123, in some embodiments, is an ancillary port system 124, which may be used to selectively deliver a fluid to and/or draw a fluid from the pericardial cavity 68. Upon initial insertion of the percutaneous access system 120 into the patient, in some embodiments, a dilator 126 may be coupled with the hub 123 and a distal end thereof may extend beyond a distal end of the introducer sheath 122. The dilator 126 thus may assist in expanding a path along which the introducer sheath 122 may be inserted into the pericardial cavity 68.

With reference to FIG. 1D, after the introducer sheath 122 is positioned within the pericardial cavity 68, the dilator 126 may be removed. An anchoring assembly 130 can then be inserted through the introducer sheath 122 into the pericardial cavity 68. The anchoring assembly 130 can be configured to interact with the magnetic guide 106 so as to be held in place, anchored, or otherwise selectively fixed to the epicardium 66, as discussed further below. A variety of configurations for the anchoring assembly 130 are possible.

In the illustrated embodiment, the anchoring assembly 130 includes a guidewire 132, a magnetic anchor 134, and a retraction wire 136. The guidewire 132 can be attached to the magnetic anchor 134 in any suitable manner that permits selective disengagement of the guidewire 132 from the magnetic anchor 134, or that otherwise temporarily attaches the guidewire 132 to the magnetic anchor 134. For example, in the illustrated embodiment, the guidewire 132 is lightly welded to the proximal end of the magnetic anchor 134 so as to permit separation of the guidewire 132 from the magnetic anchor 134 upon application of a suitable amount of force. Other disengagement techniques are also possible, depending on the temporary fastening system used. In other embodiments, the guidewire 132 may be permanently attached to the magnetic anchor 134.

The retraction wire 136 can be permanently attached to the magnetic anchor 134. For example, in the illustrated embodiment, the retraction wire 136 is welded to the distal end of the magnetic anchor 134. As further discussed below, the retraction wire 136 can remain engaged with the magnetic anchor 134 throughout a lead placement procedure, and may be used to remove the magnetic anchor 134 from the patient in the latter stages of the procedure.

In some embodiments, the guidewire 132 and the retraction wire 136 may be substantially the same. For example, the guidewire 132 and the retraction wire 136 may be formed of the same material and have the same thickness and other physical properties. In other embodiments, the guidewire 132 and the retraction wire 136 may have different properties, such as different diameters and/or different stiffnesses or flexibilities. In some embodiments, the guidewire 132 may have a slightly smaller diameter than the retraction wire 136. For example, the guidewire 132 may have a diameter of about 0.46 to about 0.91 millimeters (e.g., about 0.018 to 0.036 inches) as compared to about 0.97 millimeters (e.g., about 0.038 inches).

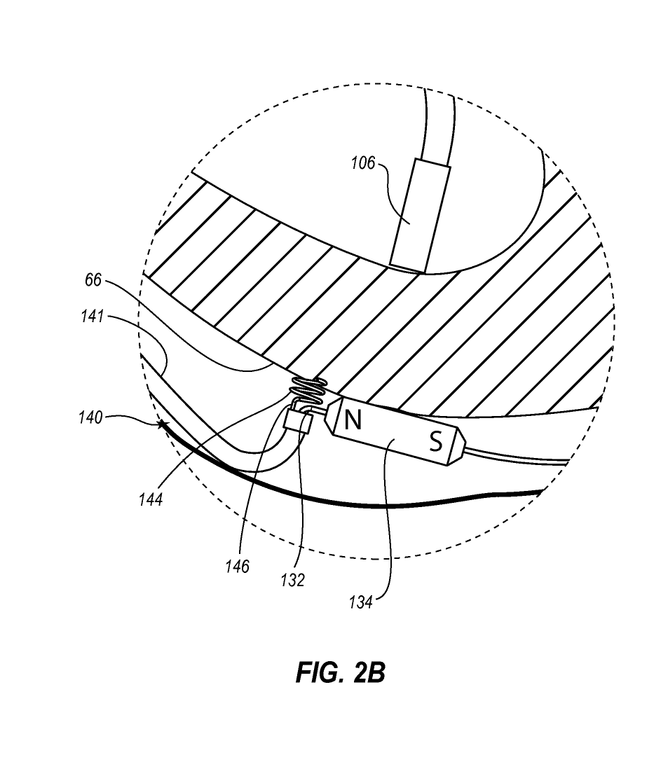

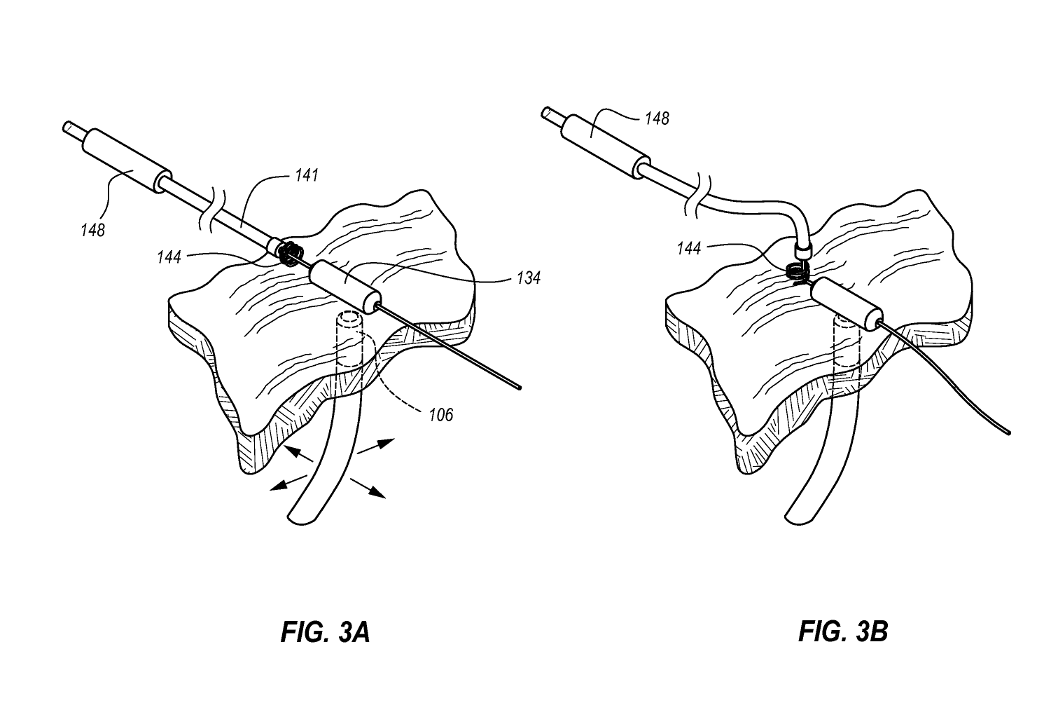

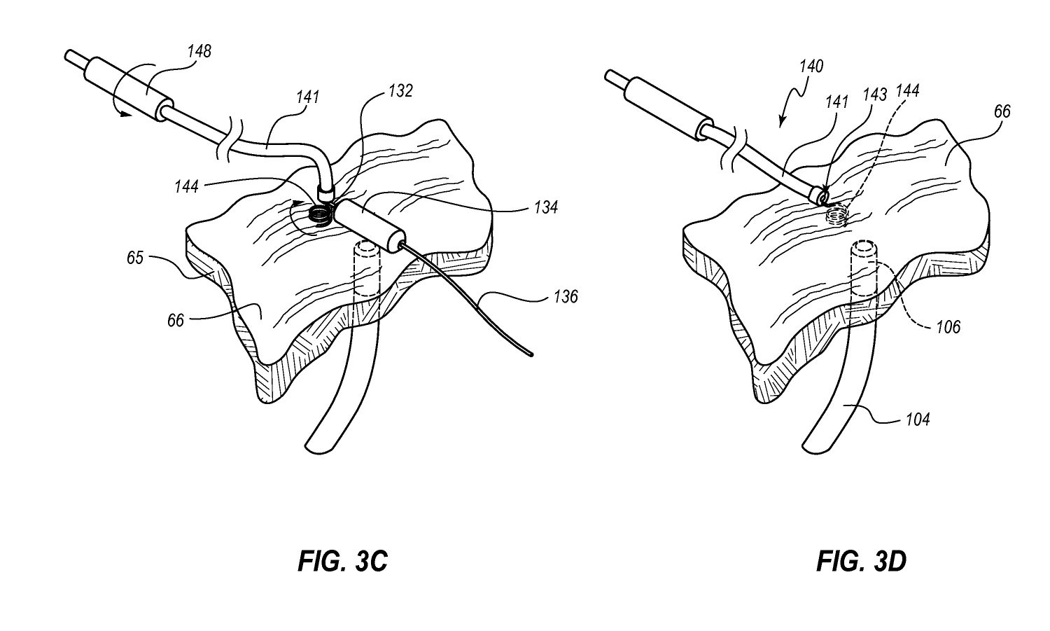

In some embodiments, the magnetic anchor 134 can have a substantially greater diameter than the diameter (or diameters) of the guidewire 132 and the retraction wire 136. For example, in various embodiments, the diameter of the magnetic anchor 134 may be no great than about 2.3, 3.0, or 3.3 millimeters, and may be capable of being inserted into the pericardial cavity 68 through an 8, 9, or 10 French introducer sheath 122, respectively. In some embodiments, the diameter of the magnetic anchor 134 may be smaller than the inner diameter of the introducer sheath 122 by an amount sufficient to permit the retraction wire 136 and the magnetic anchor 134 to be advanced distally through the sheath 122 in a side-by-side arrangement. In other or further embodiments, as discussed further below with respect to FIG. 3D, the diameter of the magnetic anchor 134 may be smaller than the inner diameter of the introducer sheath 122 by an amount sufficient to permit the magnetic anchor 134 to be retracted proximally through the sheath 122 alongside an elongated lead body 141 that has been advanced over the guidewire 132 and affixed to the epicardium 66 and that may be stationary relative to the introducer sheath 122 during removal of the magnetic anchor 134.

With continued reference to FIG. 1D, the distal portion of the anchoring assembly 130 can be advanced distally through the introducer sheath 122 into the pericardial cavity 68. In some embodiments, the retraction wire 136 extends distally relative to the magnetic anchor 134 and doubles back on itself, as shown in the FIG. 1D. In the illustrated embodiment, proximal portions of the guidewire 132 and/or the retraction wire 136 can be manipulated by a practitioner outside of the percutaneous access system 120 to effect movement of the magnetic anchor 134.

In the illustrated embodiment, each of the wires 132, 136 is sufficiently stiff to permit the magnetic anchor 134 to be advanced distally through the introducer sheath 122 by pushing on a proximal end or portion of one or more of the wires 132, 136. In further embodiments, the wires 132, 136 may be sufficiently flexible to permit at least limited movement of the magnetic anchor 134 in directions that are transverse to the longitudinal axes of the wires 132, 136, such as after the magnetic anchor 134 has exited the distal end of the introducer sheath 122 into the pericardial sack. Such flexibility may permit the magnetic anchor to readily respond to magnetic interactions with the magnetic guide 106 with little disruption from the wires.

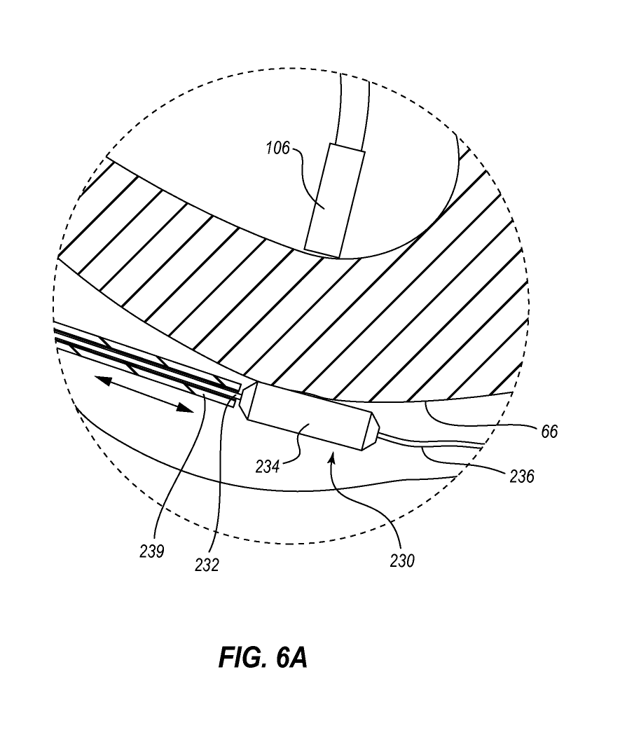

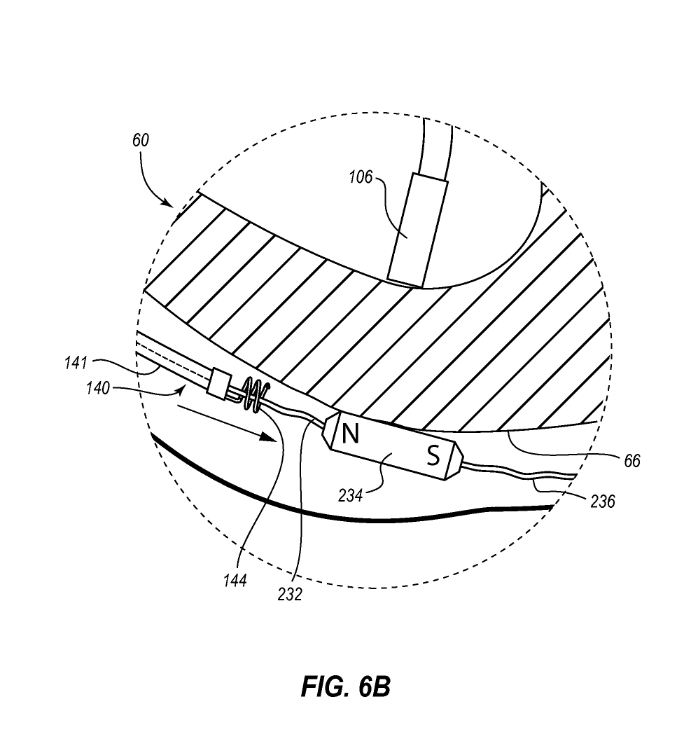

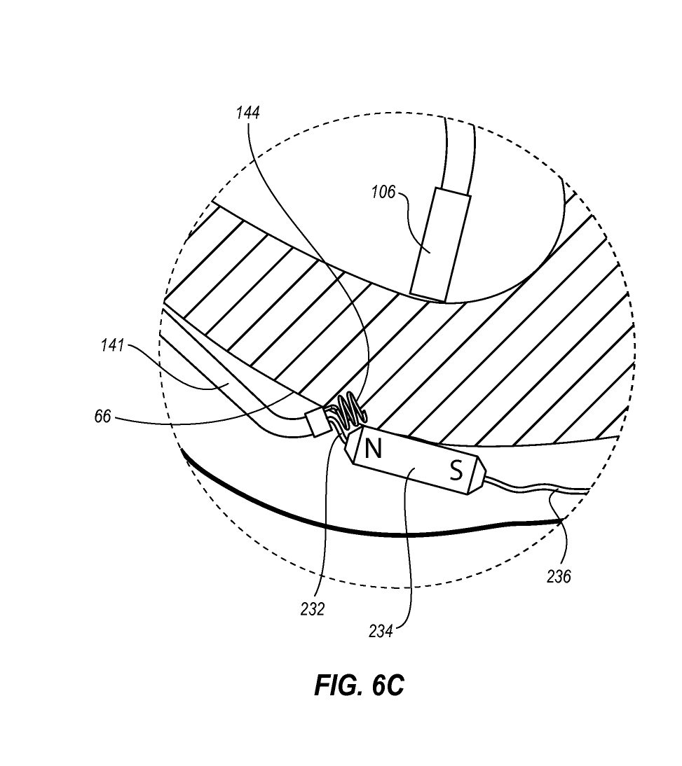

In some embodiments, only a single guidewire 132 is used. For example, in some embodiments, the guidewire 132 is used to position the magnetic anchor 134 in the pericardial sack, and is further used to retract the magnetic anchor 134 from the patient. In still other embodiments, the guidewire 132 may be significantly more flexible than the retraction wire 136, or vice versa. In still other embodiments, as discussed below with respect to FIGS. 6A-6C, the guidewire 132 and the retraction wire 136 can have little to no structural rigidity, except in tension, so as to permit even freer movement of the magnetic anchor 134 in response to movement of the magnetic guide 106 when little or no tension is applied to the guidewire 132 and the retraction wire 136.

With continued reference to the embodiment depicted in FIG. 1D, the magnetic anchor 134 can be manipulated into transmural proximity relative to the magnetic guide 106. Magnetic interaction between the magnetic guide 106 and the magnetic anchor 134 can then hold the magnetic anchor 134 against the epicardium 66. The magnetic interaction can be sufficient to cause the anchor 134 to maintain a fixed position relative to the epicardium 66. In some embodiments, the magnetic anchor 134 and the magnetic guide 106 comprise complementary magnetic sources (e.g., complementary permanent magnets). In other embodiments, one of the magnetic anchor 134 and the magnetic guide 106 comprises a magnetic source and the other of the magnetic anchor 134 and the magnetic guide 106 comprises a magnetically influenced material.

With reference to FIGS. 1D and 1E, endocardial movement of the magnetic guide 106 can effect epicardial movement of the magnetic anchor 134. The magnetic guide 106 thus can be positioned or repositioned via the steering catheter 104 to achieve a desired position of the magnetic anchor 134. As shown in FIG. 1E, in some arrangements, movement of the magnetic anchor 134 away from the distal tip of the introducer sheath 122 can draw more of the guidewire 132 distally out of the sheath 122. In some instances, it may be desirable to pull the retraction wire 136 proximally to compensate for the same.

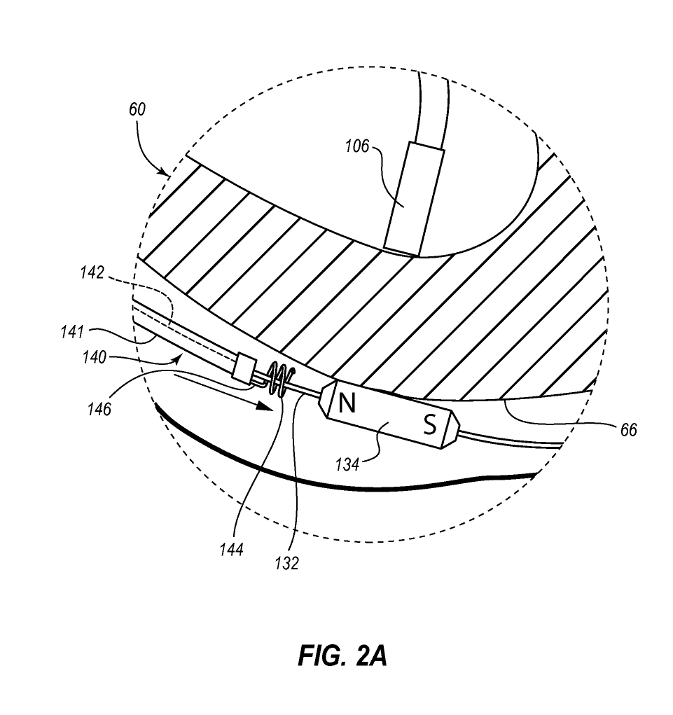

With reference to FIG. 2A, once the magnetic anchor 134 has been moved to the desired position relative to the epicardium 66, an electrical lead assembly 140 can be advanced distally over the guidewire 132 into close proximity to the magnetic anchor 134. In some arrangements, the lead assembly 140 can be advanced into contact with the magnetic anchor 134. The magnetic anchor 134 thus may act as a stop for the lead assembly 140.

In the illustrated embodiment, the lead assembly 140 comprises an elongated body 141, which may comprise any suitable catheter- or sheath-like body. In various embodiments, the elongated body 141 can be flexible and may be formed of any suitable biocompatible material. In some embodiments, the elongated body 141 defines a lumen 143 (see FIG. 3D) that is sized to receive or pass over the guidewire 132. In some embodiments, a diameter of the magnetic anchor 134 is larger than a diameter defined by the lumen 143 such that the magnetic anchor 134 can stop distal movement of the elongated body 141. In some embodiments, the lead assembly 140 can comprise any suitable pacemaker lead or other electrical lead, including varieties known in the art or yet to be devised.

In some embodiments, an electrical lead 142 is incorporated into a wall of the elongated body 141. The electrical lead 142 can be configured to transmit electrical signals from an electrical controller (not shown), such as a pacemaker or other suitable device, to the heart 60 and/or to transmit electrical signals from the heart 60 to the electrical controller. Accordingly, a proximal end of the lead assembly 140 can be configured to couple with an electrical controller in any suitable manner. For example, the proximal end of the lead assembly 140 can comprise a connector 148, which is schematically illustrated in FIGS. 3A-3E. The connector 148 may comprise one or more electrical contacts, sockets, or any other suitable electrical connection arrangements. Signals delivered to the heart 60 via the electrical lead 142 can be used for testing, pacing, and/or otherwise treating the heart. Signals received from the heart 60 via the electrical lead 142 may be used for diagnosing, positioning, mapping, and/or testing the heart.

In some embodiments, a distal end of the electrical lead 142 may be exposed so as to be able to directly contact the heart 60. In other embodiments, the electrical lead 142 may be electrically coupled with an electrical contact at the distal end of the elongated body 141, which may be electrically coupled with the heart 60 via direct contact. In still other embodiments, such as that illustrated in FIG. 2A, the electrical lead 142 may be electrically coupled with a fastener 144 that is configured to abuttingly contact the epicardial surface of the heart 60 or to be inserted into a wall of the heart 60 and fixedly coupled thereto. The fastener 144 can be electrically conductive. In the illustrated embodiment, the fastener 144 comprises a helical wire formed as a screw. The helical wire defines an axis that is collinear with the guidewire 132 in the configuration shown in FIG. 2A.

With continued reference to FIG. 2A and with additional reference to FIG. 3A, the magnetic guide 106 holds the magnetic anchor 134 tightly against the epicardium 66. As shown by an arrow (in FIG. 2A), the lead assembly 140 can be advanced distally over the guidewire 132 toward the magnetic anchor 134, of which a proximal end may be positioned at a location at which it is desirable to insert the fastener 144 into the heart 60. The lead assembly 140 can further include an actuator that can be manipulated to control movement of the fastener 144. In the illustrated embodiment, the connector 148 can be manipulated as an actuator. Accordingly, in the illustrated embodiment, the connector 148 may also be referred to as an actuator 148. In other embodiments, the lead assembly 140 may comprise an actuator that is distinct from the connector 148. In the illustrated embodiment, the actuator 148 may be situated proximally relative to the percutaneous access system 120. The percutaneous access system 120 is not shown in FIGS. 2A and 3A, but may nevertheless be present at this stage of the procedure. That is, although the percutaneous access system 120 is not shown in FIGS. 2A and 3A, it may be in substantially the same position illustrated in FIGS. 1D and 1E during this stage of the procedure. In some embodiments, a wire 146 or other coupling device links the actuator 148 to the fastener 144.

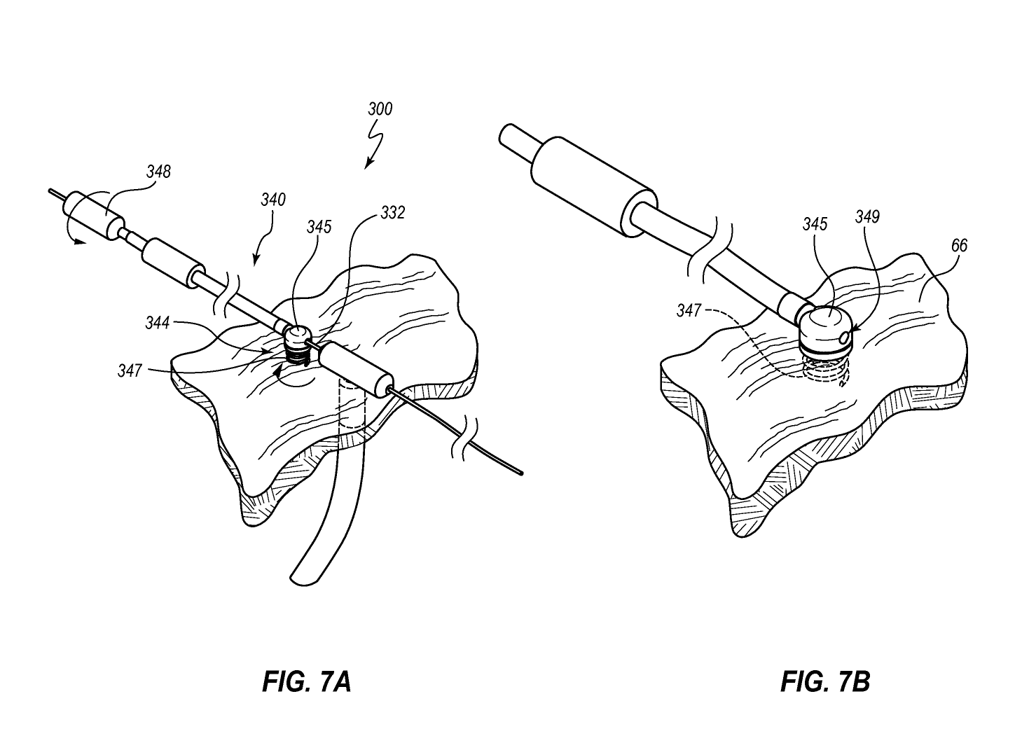

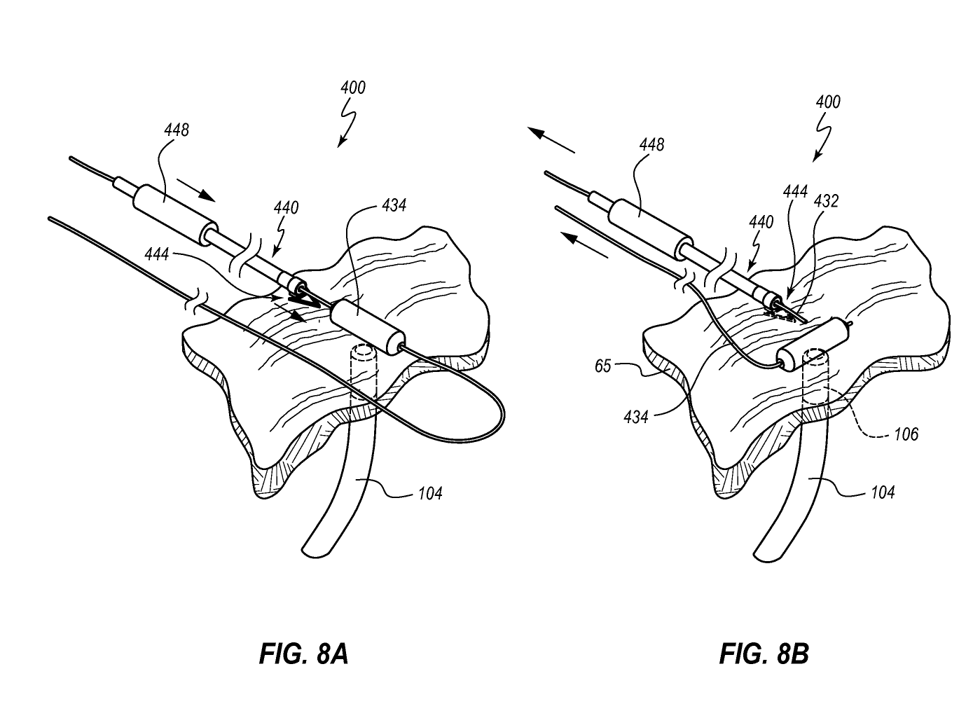

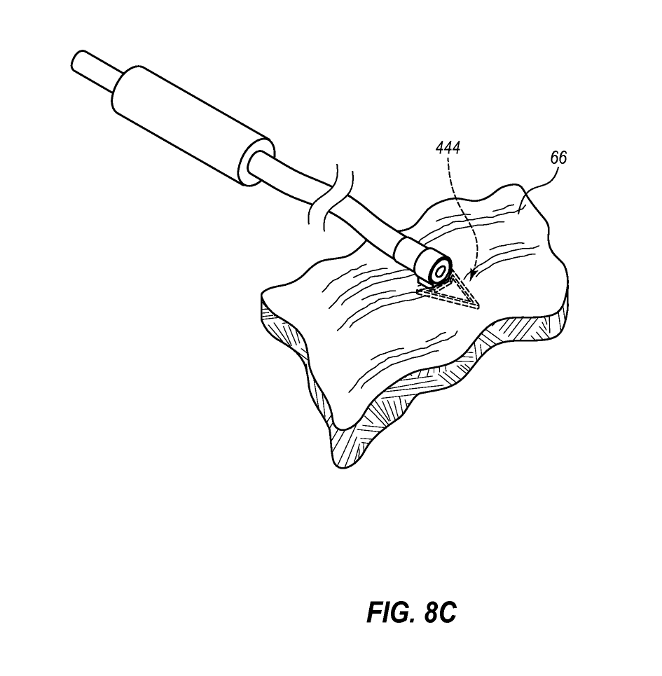

With reference to FIGS. 2B and 3B, further distal advancement of the lead assembly 140 can cause the guidewire 132 to bend. The bending may be due to counteractive forces provided by the magnetic anchor 134. For example, the magnetic interaction that keeps the magnetic anchor 134 in relatively close proximity to the magnetic guide 106 can be strong enough to resist movement of the magnetic anchor 134 away from the magnetic guide 106. This resistive force can be stronger than the bending strength of the guidewire 132. In some embodiments, the guidewire 132 may be predisposed to bend in a given direction, such as outwardly and away from the pericardium. For example, the magnetic anchor 134 may have a flattened side or other surface feature that assists in orienting the magnetic anchor 134 relative to the epicardium 66, and the guidewire 132 may be preconfigured to bend away from this feature. The guidewire 132 may, for example, be formed from a shape-memory alloy. Bending of the guidewire 132 can direct a distal tip of the fastener 144 toward the epicardium 66 so that the fastener 144 can be advanced into the wall of the heart. Any other suitable arrangement, configuration, and/or system is contemplated for attaching the fastener 144 to the heart 60 in the vicinity of the magnetic anchor 134 (see, e.g., FIGS. 7A-8B and associated discussion).

In the orientation shown in FIGS. 2B and 3B, the fastener 144 is in electrical contact with the wall of the heart, but it has not yet been advanced into the wall of the heart 60. This arrangement may be suitable for positioning and testing. For example, in some instances, it may be desirable to position the fastener 144 in electrical contact with the wall of the heart, as shown, and then determine whether the contact point is effective for the desired purpose (e.g., is an effective excitation location and/or an effective sensing location). Such a determination may be made by delivering one or more electrical signals to the heart via the electrical lead 141 or by receiving one or more electrical signals from the heart via the electrical lead 141. Where electrical signals are delivered, a reaction of the heart thereto may be monitored. If the target site meets the desired criteria, the fastener 144 may then be secured to the heart in manners such as discussed hereafter. However, if the testing reveals that the contact point is unsuitable, then fastener 144 may be relocated to another position and additional testing performed until a desired location is identified.

With reference to FIG. 3C, the fastener 144 may also be used for affixing the electrical lead assembly 140 to the heart 60. In the illustrated embodiment, rotation of the actuator 148 can effect rotation of the screw-like fastener 144, and can thereby advance the fastener 144 through the epicardium 66 into the myocardium 65.

FIG. 3D illustrates that the fastener 144 has been advanced through the epicardium 66 and secured within the myocardium 65. FIG. 3D further illustrates that after the fastener 144 has been secured, the magnetic anchor 134 can be retracted from the patient. In the illustrated embodiment, the magnetic anchor 134 is retracted by moving the retraction wire 136 proximally. For example, the proximal end of the retraction wire 136 can be pulled, which can cause the magentic anchor 134 to disengage from the magnetic guide 106. Further proximal movement of the retraction wire 136 can cause the magnetic anchor 134 to enter through the distal tip of the introducer sheath 122 (see FIGS. 1D and 1E) and move proximally relative to the elongated body 141. For example, as previously mentioned, in some embodiments, an inner diameter of the introducer sheath 122 can be larger than a combined width (e.g., a summation of the diameters) of the elongated body 141 and the magnetic anchor 134. During retraction of the magnetic anchor 134, the elongated body 141 may be positioned within the introducer sheath 122 and may be substantially stationary relative to the introducer sheath 122 as the magnetic anchor 134 is moved proximally through the introducer sheath 122 at an exterior of the elongated body 141.

As the magnetic anchor 134 is moved proximally, the guidewire 132 may follow the magnetic anchor 134. For example, a distal end of the guidewire 132 that is attached to the magnetic anchor 134 may move proximally as the magnetic anchor 134 is withdrawn from the patient. Proximal portions of the guidewire 132 that are within the lumen 143 of the elongated body 141 may thus be pulled distally, and can ultimately pass through the distal end of the lumen 143. These more proximal portions of the guidewire 132 may then reverse direction and then be pulled proximally through the introducer sheath 122 at the exterior of the elongated body 141.

In other embodiments, the guidewire 141 may interact with the elongated body 141 in a different manner. For example, in some embodiments, an external surface of the elongated body 141 may be positioned adjacent to the guidewire 132 during insertion of the magnetic anchor 134. For example, in some embodiments, the elongated body 141 may be coupled to the guidewire 132 in such a manner that the guidewire 132 is external to a substantial portion of the elongated body 141 during placement and anchoring of the magnetic anchor 134. After the fastener has been implanted, the guidewire 132 may then be decoupled from the elongated body 141, and the guidewire 132 may be retracted from the patient. In certain of such embodiments, the elongated body 141 may be devoid of a lumen and/or the guidwire 134 may not pass through the elongated body 141, whether during placement or retraction of the magnetic anchor 134. In some embodiments, the elongated body 141 may include one or more of a guide (e.g., a selectively closable loop) or other temporary fastener that is configured to selectively engage and disengage from the guidewire 132. In other embodiments, the guidewire 132 may include a temporary fastener that is configured to selectively engage and disengage from the elongated body 141.

In still other embodiments, the elongated body 141 may define a larger lumen 143 and/or the magnetic anchor 134 may have a smaller outer diameter, as compared with the illustrated arrangement, such that the guidewire 132 and the magnetic anchor 134 may be withdrawn proximally through the lumen 143 of the elongated body 141. In certain of such embodiments, the retraction wire 136 may be omitted. For example, in some embodiments, the magnetic anchor 134 may be advanced distally into the pericardial sack via the guidewire 132, and after anchoring of the fastener 144 to the wall of the heart, the guidewire 132 may be moved proximally through the elongated body 141 to also retract the magnetic anchor 134 through the elongated body 141.

FIG. 3D also depicts that, in the illustrated procedure, the steering catheter 104 is not removed from the heart prior to removal of the magnetic anchor 134. Rather, in this procedure, the retraction wire 136 is pulled to disengage the magnetic anchor 134 from the magnetic guide 106. Thereafter, the steering catheter 104 and the magnetic guide 106 can be removed through the vasculature of the patient in any suitable manner. In other procedures, the magnetic guide 106 may be retracted from the endocardial wall of the heart to reduce the magnetic interaction between the magnetic guide 106 and the magnetic anchor 134, which may facilitate removal of the magnetic anchor 134. For example, in some procedures, the magnetic guide 106 may be retracted from the endocardial wall before the retraction wire 136 is used to withdraw the magnetic anchor 134 from the patient. In still other embodiments, the magnetic guide 106 and the magnetic anchor 134 may be retracted from the patient substantially concurrently.

FIG. 4 illustrates an embodiment of the guiding assembly 102. In the illustrated embodiment, the guiding assembly 102 can be used at endocardial positions. Accordingly, the guiding assembly 102 may also be referred to herein as an endocardial assembly. The guiding assembly 102 can include the magnetic guide 106 and the steering catheter 104.

The steering catheter 104 of the guiding assembly 102 can comprise any suitable probe or catheter arrangement. For example, any suitable steering catheter arrangement is possible, such as any of the steering catheter arrangements are disclosed in U.S. Pat. No. 7,938,828, titled COOLED ABLATION CATHETER, which issued on May 10, 2011, the entire contents of which are hereby incorporated by reference herein. The steering catheter 104 can aid a practitioner in positioning the magnetic guide 106 at a desired location and in a desired orientation within the patient, and/or may aid in movement of the magnetic guide 106 during a procedure.

The guiding assembly 102 can include a steering assembly 150 that is configured to effect movement of the magnetic guide 106 at the distal end of the catheter 104. The steering assembly 150 can include a handle 151, a steering lever 152, and a locking lever 154.

With reference to FIGS. 5A-5C, the steering assembly 150 can further include one or more steering wires 172 and a spring element 174. FIGS. 5A-5C depict cross-sectional views of a distal end of the catheter 104 in various orientations. For the sake of clarity, various features of the catheter 104 that are not part of the steering assembly 150 are not shown in these views. The spring element 174 can be attached to or otherwise coupled with a sheath 170 portion of the catheter 104, such that movement of the spring element 174 effects a corresponding movement of the catheter 104, and such that an orientation of the spring element 174 results in a corresponding orientation of the catheter 104. Other arrangements are also possible, including arrangements that might not employ a spring element.

In the illustrated embodiment, the steering assembly 150 includes a single steering wire 172 that is connected to the steering lever 152 at a proximal end thereof and that is connected to one side of the spring element 174 at a distal end thereof. In use, the steering lever 152 can be rotated relative to the handle 151 so as to move the steering wire 172 relative to the spring element 174. The locking lever 154 may be used to fix a position of the steering lever 152 relative to the handle 151, and thereby fix a position of the steering wire 172 relative to the spring element 174 so as to maintain a desired configuration of the distal end of the catheter 104. Orientation of the catheter 104 in this manner can be used to provide a desired orientation of the magnetic guide 106. Other embodiments can include two steering wires 172, with each wire being connected to opposite sides of the spring element 174.

FIG. 5A illustrates a natural or relaxed orientation of the catheter 104 in which the steering wire 172 neither compresses nor extends one side of the spring element 174. In such an arrangement, the catheter 104 may be substantially linear.

FIG. 5B illustrates an orientation to which the catheter 104 can be transitioned by rotating the steering lever 152 so as to move the steering wire 172 in the proximal direction, as illustrated by the arrow. Such movement can compress an upper side of the spring element 174, causing the catheter 104 to curve in a first direction. In such an orientation, the catheter 104 may be substantially J-shaped.

FIG. 5C illustrates an orientation to which the catheter 104 can be transitioned by rotating the steering lever 152 so as to move the steering wire 172 in the distal direction, as illustrated by the arrow. Such movement can extend an upper side of the spring element 174, causing the catheter 104 to curve in a second direction that is opposite the first direction depicted in FIG. 5B. The catheter 104 may again define a J-shape in this orientation. Any other suitable steering catheter arrangement is contemplated. Arrangements for the steering catheter 104 other than those just described are also contemplated. The steering catheter 104 can be configured to move freely through three dimensions.

With reference again to FIG. 4, in some embodiments, the guiding assembly 102 includes one or more controllers 160 that can be electrically and/or fluidly coupled with the steering assembly 150 and/or the catheter 104 via one or more cables or conduits 162. Auxiliary functions that may be performed by the one or more controllers include electrical monitoring, delivery of electrical charges, control of a strength of a magnetic field produced by the magnetic guide 106 (such as when the guide 106 comprises an electromagnet), and/or cooling of the magnetic guide 106.

FIGS. 6A-6C illustrate another embodiment of a system 200 that can resemble the system 100 described above in certain respects. Accordingly, like features are designated with like reference numerals, with the leading digits incremented to “2.” Relevant disclosure set forth above regarding similarly identified features thus may not be repeated hereafter. Moreover, specific features of the system 200, may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the system 200. Any suitable combination of the features and variations of the same described with respect to the system 100 can be employed with the system 200, and vice versa. This pattern of disclosure applies equally to further embodiments depicted in subsequent figures and described hereafter, wherein the leading digits may be further incremented.

The system 200 can include an anchoring assembly 230 similar to the anchoring assembly 130 discussed above. In the illustrated embodiment, the anchoring assembly 230 includes a guidewire 232, a magnetic anchor 234, and a retraction wire 236. The guidewire 232 and the retraction wire 236 can be tether-like structures with little or no rigidity in directions transverse to the longitudinal axes of the wires (e.g., axes defined when the guidewire 232 and the retraction wire 236 are fully extended). Stated otherwise, the guidewire 232 and the retraction wire 236 can be tethers or cables that are limp, string-like, flexible, or otherwise readily movable in transverse directions. The guidewire 232 and the retraction wire 236 can nevertheless provide tension to either end of the magnetic anchor 234 when the wires are pulled in a proximal direction.

The relative lack of rigidity of the wires can permit the magnetic anchor 234 to more freely move as a result of magnetic interactions with the magnetic guide 106, as the guidewire 232 and the retraction wire 236 provide little resistance to such movement. Stated otherwise, the guidewire 232 and the retraction wire 236 can provide very little loading to the magnetic anchor 234 that would hinder its movement in response to the magnetic guide 106. Such an arrangement can be particularly advantageous in situations where the forces provided by the magnetic guide 106 are relatively weak.

The anchoring assembly 230 can further include a positioning device 239, such as a tube or other suitable structure. In some embodiments, the positioning device 239 may be stiff, whereas in other embodiments the positioning device 239 may be relatively flexible. In either case, the positioning device 239 can be sufficiently rigid to push the magnetic anchor 234 proximally through the introducer sheath 122 (see FIGS. 1D and 1E) into proximity with the epicardium 66 of the heart and then position the magnetic anchor 234 in an area where it can be magnetically coupled to the magnetic guide 106 by manipulating and steering the positioning the steering device 239. For example, the positioning device 239 can be stiffer than the guidewire 232 and the retraction wire 236 in one or more directions that are transverse to longitudinal axes defined by the positioning device 239, the guidewire 232, and the retraction wire 236 (e.g., axes defined when these elements are fully extended). In the illustrated embodiment, the positioning device 239 comprises a tube that defines a lumen, and the guidewire 232 extends through the lumen.

In certain embodiments, the magnetic anchor 234 can be held in contact with a distal end of the positioning device 239 as the magnetic anchor 234 is advanced distally into the pericardial sack and then positioned in an area in which the magnetic anchor 234 can magnetically interact with the magnetic guide 106. For example, in some embodiments, a limited amount of tension is provided on the guidewire 232 in a proximal direction relative to the positioning device 239 to maintain the contact between the magnetic anchor 234 and the distal end of the positioning device 239.

In some embodiments, the positioning device 239 may be retracted proximally over the guidewire 232 after the magnetic anchor 234 has been magnetically coupled with the magnetic guide 106. The magnetic interaction between the magnetic anchor 234 and the magnetic guide 106 can maintain the magnetic anchor 234 in a generally fixed position relative to the flexible guidewire 232, such that the positioning device 239 can be retracted proximally over the guidewire 232 as the magnetic anchor 234 remains in place. The magnetic anchor 234 can then move freely in response to movement of the magnetic guide 106.

With reference to FIG. 6B, in some embodiments, after the positioning device 239 has been retracted proximally over the guidewire 232, the elongated body 141 of the electrical lead assembly 140 is advanced distally over the guidewire 232. In some embodiments, a small amount of tension may be provided on the guidewire 232 while the elongated body 141 is advanced over the guidewire 232. In such instances, the amount of tension provided is insufficient to decouple the magnetic anchor 234 for the magnetic guide 106. The fastener 144 of the electrical lead assembly 140 can be urged into proximity to the magnetic anchor 234.

In other embodiments, rather than retracting the positioning device 239, the electrical lead assembly 140 can be advanced over the positioning device 239. For example, the flexible guidewire 232 may remain within the positioning device 239, and the elongated body 141 may be advanced over the positioning device 239. However, in certain of such arrangements, the presence of the positioning device 239 may hinder movement of the magnetic anchor 234.

With reference to FIG. 6C, the fastener 144 can be advanced into the wall of the heart in manners such as described above. In some instances, there may be less interaction between the fastener 144 and the guidewire 232 during such an anchoring event due to the flexibility of the guidewire 232.

After anchoring of the fastener, in some embodiments, the guidewire 232 may be removed from the magnetic anchor 234, such as in manners discussed above. Accordingly, in some embodiments, the magnetic anchor 234 may be retracted from the patient via the retraction wire 236, and may pass alongside an exterior of the elongated body 141 in a proximal direction, whereas the guidewire 232 may be retracted from the patient separately by being moved proximally through the elongated body 141.

In other embodiments, the guidewire 232 is permanently attached to the magnetic anchor 234. As previously discussed, in the illustrated embodiment, the guidewire 232 may be limp, string-like, or otherwise highly flexible. In certain of such embodiments, the guidewire 232 may be retracted from the patient by pulling proximally on the retraction wire 236. This can cause the guidewire 232 to move distally through the elongated body 141 and then, after exiting the elongated body 141, move proximally through the introducer sheath 122 (see FIGS. 1D and 1E) so as to be retracted from the patient.

FIGS. 7A and 7B illustrate another embodiment of a system 300 such as the systems 100, 200 discussed above. The system 300 includes an electrical lead assembly 340 that functions in a manner somewhat different from the electrical lead assembly 140. The electrical lead assembly 340 includes a fastener 344 that includes a housing 345 and a screw 347. The housing 345 defines a lumen 349 through which a guidewire 332 can pass.

An axis of the screw 347 is offset from an axis defined by the guidewire 332. In the illustrated embodiment, these axes are perpendicular to each other. Rotation of an actuator 348 effects rotation of the screw 347 for advancement into the epicardium 66.

In some embodiments, the electrical lead assembly 340 can comprise a system such as that disclosed in U.S. Pat. No. 4,357,946, titled EPICARDIAL PACING LEAD WITH STYLET CONTROLLED HELICAL FIXATION SCREW, which issued on Nov. 9, 1982, the entire contents of which are hereby incorporated by reference herein.

FIGS. 8A-8C illustrate another embodiment of a system 400 such as the systems 100, 200, 300 discussed above. The system 400 includes an electrical lead assembly 440 that functions in a manner somewhat different from the electrical lead assemblies 140, 340.

With reference to FIG. 8A, the electrical lead assembly 440 includes a fastener 444 having a sharp end. The fastener 444 can be translated distally into contact with a magnetic anchor 434. In some embodiments, a proximal end of the magnetic anchor 434 can deflect the sharp end of the fastener 444 into the myocardium 65. An actuator 448, which may merely comprise a gripping pad or gripping region in some embodiments, or may include one or more electrical connectors in other embodiments, can be used to advance the fastener 444 distally.

With reference to FIG. 8B, in some embodiments, once the fastener 444 has been secured, the guidewire 432 can be separated from the magnetic anchor 434. In some embodiments, this separation is achieved by twisting the guidewire 432 and/or by repeatedly pushing and pulling on the guidewire 432 to weaken the point of attachment between the guidewire 432 and the magnetic anchor 434. In some embodiments, the magnetic forces between the magnetic anchor 432 and the magnetic guide 106 are strong enough to counteract such manipulations of the guidewire 432. Other methods for disengaging the guidewire 432 from the magnetic anchor 434 are also possible. Once separation is achieved, a retraction wire 436 can be withdrawn proximally from the patient to retract the magnetic anchor 434. The guidewire 432 may be withdrawn proximally through a lumen of the electrical lead assembly (such as the lumen 143 discussed above).

FIG. 8B illustrates a point in time after separation of the guidewire 434 from the magnetic anchor 434 has been achieved. Tension is provided on the retraction wire 436, as illustrated by a proximally directed arrow, to disengage the magnetic anchor 434 from the magnetic guide 106 and then remove the magnetic anchor 434. In the illustrated embodiment, the retraction wire 436 is pulled proximally through an introducer sheath (such as the introducer sheath 122 in FIGS. 1D and 1E), and the magnetic anchor 434 trails the retraction wire 436, due to the permanent attachment of these components to each other. As with certain embodiments discussed above, in some embodiments, the maximum transverse diameter of the magnetic anchor 434 is smaller than the inner diameter of the introducer sheath by an amount sufficient to permit the magnetic anchor 434 to be retracted proximally through the sheath 422 alongside an elongated lead body. As the elongated body has been affixed to the heart wall via the fastener 444, the elongated body can be substantially stationary relative to the introducer sheath during removal of the magnetic anchor 434.

FIG. 8C illustrates a point in time after the retraction wire 436 and the magnetic anchor 434 have been withdrawn and after withdrawal of the guidewire 432. Whether before, after, or concurrently with the removal of the magnetic anchor 434, discussed above, the guidewire 432 may be removed from the patient. The guidewire 432 can be retracted proximally through the lumen defined by the elongated body portion of the electrical lead assembly 440. In the illustrated embodiment, an inner diameter of the lumen is substantially smaller than the outer diameter of the magnetic anchor 434, which can prevent the magnetic anchor 434 from being retracted through the elongated body. Accordingly, separation of the guidewire 432 from the magnetic anchor 434 for purposes of retraction can be advantageous.

In some instances, the magnetic guide 106 may be retracted from the endocardial wall of the heart to reduce the magnetic interaction between the magnetic guide 106 and the magnetic anchor 434, which may facilitate removal of the magnetic anchor 434. For example, in some procedures, the magnetic guide 106 may be retracted from the endocardial wall after the magnetic anchor 434 has been separated from the guidewire 432, but before the magnetic anchor 434 has been withdrawn via the retraction wire 436 (e.g., at a stage such as that shown in FIG. 8B)

Although much of the foregoing disclosure is directed to various stages of cardiac resynchronization therapy, the various embodiments discussed, or features thereof, are not necessarily limited to any specific procedure or context. The apparatus, systems, and methods disclosed herein may be applied to any surgical procedure that can benefit from, for example, temporary fixation of an anchoring device via magnetic interaction between elements that are separated from each other by a tissue mass, such as for the purpose of electrically mapping, electrically actuating, or otherwise treating or interacting with the tissue. Other or further features of various embodiments disclosed herein may also be applicable in different contexts. Applications of various embodiments include, but are not limited to, placement of catheters, pacemakers, imaging devices, biosensors, and/or prostheses, and/or observation or measurement of electrical properties of tissue. For example, although certain embodiments of pacing leads are shown and described for anti-bradycardia or resynchronization therapies, the disclosure can also apply to epicardial leads that deliver to the patient's heart high energy electrical shocks for cardioversion/defibrillation to alleviate or terminate a tachyarrhythmia.

Moreover, in other or further embodiments, a magnetic guide may be positioned at an interior of any suitable anatomical structure, such as a walled cavity or vessel, and a fastener or other anchoring device can be secured at an exterior of the anatomical structure in manners such as described herein. Similarly, in other or further embodiments, a guide may be positioned at an exterior of the anatomical structure and an anchoring device may be secured to an interior of the structure. Accordingly, although much of the present disclosure is provided in the context of cardiac procedures, certain systems, apparatus, and methods can be configured for and/or used in other contexts.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “about” or “approximately.” For each such reference, it is to be understood that, in some embodiments, the value, feature, or characteristic may be specified without approximation. For example, where qualifiers such as “about,” “substantially,” and “generally” are used, these terms include within their scope the qualified words in the absence of their qualifiers. For example, where the term “substantially linear” is recited with respect to a feature, it is understood that in further embodiments, the feature can have a precisely linear configuration.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. Recitation in the claims of the term “first” with respect to a feature or element does not necessarily imply the existence of a second or additional such feature or element.

1. A system for use with a heart having a pericardium, an epicardium, and an endocardium, the system comprising:
an anchoring assembly that comprises a guidewire and a magnetic anchor, wherein at least a distal portion of the anchoring assembly is sized to be inserted between the pericardium and the epicardium, and wherein the magnetic anchor is configured to magnetically interact with a magnetic guide that is positioned at an interior of the endocardium to hold the anchoring assembly against the epicardium; and
an elongated body that comprises an electrical lead, the elongated body having a proximal end and a distal end, wherein the proximal end is configured to be coupled with an electrical controller, and wherein the elongated body is configured to be selectively coupled with the guidewire to permit the distal end to be advanced distally relative to the guidewire into close proximity to the magnetic anchor when the magnetic anchor is between the pericardium and the epicardium.

2. The system of claim 1, wherein the elongated body defines a lumen that extends along a longitudinal length of the elongated body, and wherein the lumen is sized to receive the guidewire to permit the elongated body to be advanced over the guidewire.

3. The system of claim 2, wherein the anchoring assembly is configured to split apart after the fastener is attached to the elongated body to permit at least a portion of the guidewire that is unattached to the magnetic anchor to be withdrawn through the lumen of the elongated body.

4. The system of claim 2, wherein a diameter of the magnetic anchor is greater than an inner diameter of the lumen of the elongated body to prevent the magnetic anchor from passing through the lumen of the elongated body.

5. The system of claim 1, further comprising a fastener coupled to the distal end of the elongated body, wherein the fastener is configured to attach the elongated body to the epicardium of the heart.

6. The system of claim 5, wherein the fastener comprises a screw.

7. The system of claim 5, wherein the fastener is electrically conductive and is electrically coupled with the elongated body.

8. The system of claim 1, further comprising the magnetic guide.

9. The system of claim 8, wherein the magnetic guide comprises a steering catheter.

10. The system of claim 8, wherein one or more of the magnetic anchor and the magnetic guide comprises a permanent magnet.

11. The system of claim 1, further comprising an introducer sheath having an inner surface that defines a lumen, wherein the inner surface has an inner diameter greater than an outer diameter of the magnetic anchor and greater than an outer diameter of the elongated body to permit each of the magnetic anchor and the elongated body to be advanced through the lumen of the introducer sheath.

12. The system of claim 1, further comprising an elongated positioning device configured to advance the magnetic anchor distally to insert the magnetic anchor between the pericardium and the epicardium.

13. The system of claim 12, wherein the positioning device has a stiffness that is greater than a stiffness of the guidewire in one or more directions that are transverse to longitudinal axes defined by the guidewire and the positioning device.

14. The system of claim 12, wherein the guidewire is positioned within a lumen defined by the positioning device.

15. A method of electrically communicating with a heart having an endocardium, an epicardium, and a pericardium, the method comprising:
positioning a magnetic guide at an interior of the endocardium;
inserting a magnetic anchor that is attached to a guidewire into a space between the pericardium and the epicardium;
holding the magnetic anchor against the epicardium at a first position via magnetic interaction between the magnetic guide and the magnetic anchor;
advancing an elongated body that comprises an electrical lead along the guidewire and into the space between the pericardium and the epicardium; and
electrically coupling the electrical lead with the heart.

16. The method of claim 15, further comprising:
moving the magnetic guide relative to the endocardium to effect movement of the magnetic anchor from the first position to a second position; and
holding the magnetic anchor against the epicardium at the second position via the magnetic interaction between the magnetic guide and the magnetic anchor.

17. The method of claim 15, further comprising delivering one or more electrical signals to the heart via the electrical lead.

18. The method of claim 17, further comprising determining whether to affix the electrical lead to the heart based on a reaction of the heart to the one or more electrical signals thus delivered.

19. The method of claim 18, further comprising affixing the electrical lead to the heart while the magnetic anchor is held at the first position.

20. (canceled)

21. The method of claim 15, wherein electrically coupling the electrical lead with the heart comprises affixing a fastener into the epicardium.

22-28. (canceled)
